(19) United States
(12) Patent Application Publication
(10) Pub. No.: US 2022/0273440 A1
(43) Pub. Date: Sep. 1, 2022

(54) SYNTHETIC TISSUE-GRAFT SCAFFOLD

(71) Applicant: OREGON HEALTH & SCIENCE UNIVERSITY, Portland, OR (US)

(72) Inventors: Luiz E. BERTASSONI, Portland, OR (US); Avathamsa ATHIRASALA, Portland, OR (US); Anthony TAHAYERI, Portland, OR (US)

(21) Appl. No.: 17/597,818

(22) PCT Filed: Jan. 30, 2020
(86) PCT No.: PCT/US2020/015952

§ 371 Date: Jan. 25, 2022

Related U.S. Application Data
(63) Continuation-in-part of application No. PCT/US19/43798, filed on Jul. 26, 2019.

(51) Int. Cl. A61F 2/28; A61L 27/12; A61L 27/52; A61L 27/54

(52) U.S. Cl. CPC A61F 2/2846; A61L 27/12; A61L 27/52; A61L 27/54; A61F 2002/30985

(57) ABSTRACT

A synthetic tissue-graft scaffold (10) includes one or more nominally identical scaffold cages (12) that are configured to facilitate regrowth of tissue of an organism in and around the scaffold cages. Each scaffold cage comprises a volumetric enclosure (14) bounded by a perforated wall structure (30) that has an interior surface (32) and an exterior surface (34). A first annular inlet (22) and second annular inlet (24) positioned at opposite ends of the enclosure form, respectively, a first conjoining surface (54) and a second conjoining surface (56) that are configured so that confronting conjoining surfaces form complementary surfaces to each other. A perforated platform (60) is bounded by the interior surface of the enclosure and provides passageways (62) within the interior chamber. Corridors (40) extend through the perforated wall structure and communicate with the passageways to enable migration of material within and out of the cage.

### FEDERALLY SPONSORED RESEARCH OR DEVELOPMENT

This invention was made with government support under Grant Number NIH R01 DE026170 awarded by the National Institutes of Health. The government has certain rights in the invention.

### COPYRIGHT NOTICE

© 2020 Oregon Health & Science University. A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR § 1.71(d).

### TECHNICAL FIELD

Generally, the field involves methods for generating scaffold structures for tissue regeneration applications. More specifically, the field involves generation of synthetic scaffold cages that may be combined into larger scaffold structures. The synthetic scaffold cages are engineered to contain interconnected porous spaces into which a substrate such as a gel, including a hydrogel, may be introduced to produce a two-phase scaffold structure.

### BACKGROUND INFORMATION

Additive manufacturing (such as 3D-printing technology) has enabled significant progress in tissue-graft scaffold design and fabrication for regenerative medicine applications. This includes the capability to selectively fabricate patient-specific scaffolds of suitable shape, size, and three-dimensional complexity to support tissue regeneration of that patient's tissue defects.

Bone repair is one example of tissue regeneration that entails use of tissue-graft scaffolds. Approximately one in two adults is affected by some form of bone or musculoskeletal condition worldwide, which is twice the rate of heart and lung diseases. Craniotomies and other craniofacial procedures, such as vertical and horizontal bone augmentation, have an estimated cost of about $950 million each year, and it is estimated that more than 500,000 bone grafting procedures are conducted annually in the U.S. alone. Despite important limitations associated with autologous bone harvesting, such as the high hospitalization costs, and donor-site morbidity, bone autografts remain the gold-standard material to treat critical-sized bone defects. Therefore, it is frequently proposed that the ideal bone scaffold would match the key hallmarks of the native bone, while bypassing the challenges associated with its surgical extraction.

Although much progress has been made in the development of synthetic bone grafts, only 30% of treated patients regain function without the need for a secondary procedure, and graft failure rates can be as high as 50%. Autologous bone grafts are more successful compared to synthetic bone grafts due to their inherent vasculature, which is always present in autologous bone yet generally absent in synthetic scaffolds, and the failure of synthetic scaffolds to mimic the complexity of the cell-rich and nano-mineralized microenvironment that autologous bone provides. The native bone matrix consists of an osteocyte-laden, densely mineralized organic scaffold, where mineralization of ionic calcium and phosphorous is orchestrated on a nanometer scale, thereby resulting in a hierarchical architecture that is known to be key for bone's physical properties. Moreover, osteocytes embedded within this Calcium-and-Phosphorus-ion-rich (CaP-rich) milieu are known to control the process of bone remodeling from the “inside-out” and regulate its remodeling by secreting chemokines that attract host cells to the site of repair.

However, none of these key features are present in clinically available synthetic bone-graft scaffolds. Moreover, the reconstruction of large volume defects with autologous bone grafts remains a challenge; donor site morbidity limits the size of the harvested bone. Clinically available synthetic bone-graft scaffolds are typically composed of brittle pre-calcified ceramics or soft CaP-rich composites that rely on tissue ingrowth upon implantation for clinical success. A suitable tissue-graft scaffold would ideally include the ability to selectively compartmentalize generative tissue-graft material and allow for compartment-to-compartment migration of the material within and out of the scaffold. Moreover, treatment of large volume defects with synthetic tissue-graft scaffolds would ideally utilize a scaffold that is selectively scalable to the size and shape of the defect while having sufficient flexural strength to resist deformation after implantation.

### SUMMARY OF THE DISCLOSURE

The disclosed materials and methods relate to building models that are useful as scaffolds for tissue regeneration, such as for natural bone repair following trauma or surgery. Some of the disclosed embodiments use building blocks that are capable of forming a customized bone replacement scaffold for a portion of bone removed by surgery or trauma. A preferred synthetic tissue-graft scaffold includes a set of one or more nominally identical scaffold cages that are configured to facilitate regrowth of tissue of an organism in and around the scaffold cages. Each scaffold cage in the set comprises a volumetric enclosure bounded by a perforated wall structure and has interior and exterior surfaces and first and second opposite ends. The volumetric enclosure defines a central longitudinal axis that extends through the first and second opposite ends of scaffold cages. The interior surface defines a boundary of an interior chamber of the volumetric enclosure, and the interior and exterior surfaces define between them a thickness of the perforated wall structure. First and second annular inlets are positioned at, respectively, the first and second ends of the volumetric enclosure and form, respectively, first and second conjoining surfaces that are transverse to the central longitudinal axis. The first and second conjoining surfaces are configured so that, whenever confronting annular inlets of a pair of the scaffold cages in the set are conjoined, confronting ones of the first or second conjoining surfaces of the pair of scaffold cages form complementary surfaces to each other. A perforated platform is bounded by the interior surfaces of the volumetric enclosure and set in transverse relation to the central longitudinal axis. The perforated platform provides a passageway within the interior chamber of the volumetric enclosure between its first and second opposite ends. Corridors extend through the thickness of the perforated wall structure and communicate with the passageway within the interior chamber of the volumetric enclosure to enable migration of material within and out of the scaffold cage. A suitable synthetic scaffold may have its first and second annular inlets positioned, respectively, at the second and first opposite ends of the scaffold cage.

A suitable synthetic bone-graft scaffold built with the disclosed scaffold cages may be made of a CaP-rich composite such as high-density β-tricalcium phosphate (β-TCP). Tissue-graft scaffolds fabricated with β-TCP have sufficient flexural strength to be printed as a permeable structure that nonetheless is resistant to deformation after implantation. Moreover, natural dissolution of the β-TCP postimplantation distributes osteoinductive, ionic calcium and phosphorous into the repair-site milieu while integrating the bone-graft scaffold into the surrounding tissue. A permeable β-TCP bone-graft scaffold is preferable for allowing the selective loading of tissue-graft material into the scaffold, and for allowing for movement of the tissue-graft material throughout the scaffold and host-insertion site, thus facilitating vascularization and tissue ingrowth within the bone graft.

Additive manufacturing methods would lend themselves well to fabricating synthetic microscale scaffolds. A synthetic scaffold enables significant scalability, allowing a user to employ as many scaffold modules as needed to fill the volume of a defect. A synthetic scaffold design also allows for a selective three-dimensional assembly of the scaffold to fit the three-dimensional shape of a defect. Moreover, a selective three-dimensional assembly utilizing micro-scale modules allows a user to employ a scaffold of heterogeneous tissue-graft material composition that is specific to the defect site and clinically meaningful.

Thus, a tissue-graft scaffold fabricated by additive manufacturing methods, employing a permeable structural design, loadable with microscale, site-specific, tissue-appropriate tissue-graft materials would be suitable for constructing patient-specific synthetic tissue-graft implants.

Additional aspects and advantages will be apparent from the following detailed description of preferred embodiments, which proceeds with reference to the accompanying drawings.

### BRIEF DESCRIPTION OF THE DRAWINGS

FIG. 1 is an oblique isometric view showing a synthetic tissue-graft scaffold that includes a set of one or more nominally identical scaffold cages.

FIG. 2A1 is an oblique isometric view showing an annular inlet, and FIG. 2A2 is an oblique isometric view showing an opposite annular inlet of one embodiment of the disclosed scaffold cage.

FIG. 2B1 is a sectional view taken along lines 2B1-2B1 of FIG. 2A1, and FIG. 2B2 is a sectional view taken along lines 2B2-2B2 of FIG. 2A2 showing an interior chamber and a perforated platform of the scaffold cage of FIGS. 2A1 and 2A2.

FIGS. 3A and 3B are, respectively, top and bottom plan views of the scaffold cage of FIGS. 2A1 and 2A2.

FIG. 3C is an oblique isometric view of one embodiment of a scaffold cage configured to have a perforated platform positioned against an annular inlet of the disclosed scaffold cage.

FIG. 4 shows size and feature dimensions superimposed on a side elevation view of the scaffold cage of FIG. 1.

FIGS. 5A, 5B, and 5C are fragmentary oblique isometric views showing, respectively, first, second, and third alternative embodiments of a conjoining surface.

FIG. 6. is a cross-sectional isometric view of a 3×3 synthetic tissue-graft scaffold cage sheet formed by fusing in a 3×3 arrangement nine replicas of the scaffold cage of FIGS. 2A1 and 2A2.

FIGS. 7A and 7B are respective exploded and isometric views of three scaffold cages arranged for assembly to construct a scaffold cage tier.

FIG. 7C is a cross-sectional isometric view of a scaffold cage tier taken along lines 7C-7C of 7B.

FIG. 8 is a cross-sectional isometric view of two 3×1 cage tiers as exemplified in FIG. 7C having a fused perforated wall structure to form a tiered cage sheet.

FIG. 9 is an isometric view of one 4×1 and two 3×1 cage tiers assembled to form a tiered cage sheet having multiple fused perforated wall structures.

### DETAILED DESCRIPTION OF PREFERRED EMBODIMENTS

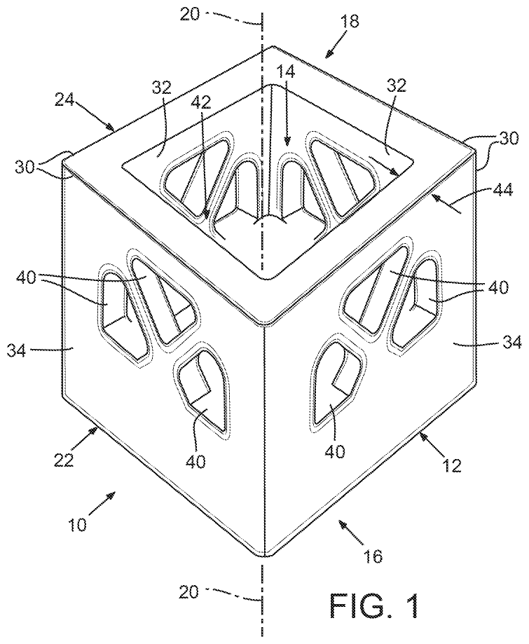

FIG. 1 is an oblique isometric view showing an example of a synthetic tissue-graft scaffold 10 that includes a set of one or more nominally identical scaffold cages 12. In the example shown, synthetic tissue-graft scaffold 10 includes a set of one scaffold cage 12. As shown in FIG. 1, scaffold cage 12 includes a volumetric enclosure 14 having a first opposite end 16 and a second opposite end 18. Volumetric enclosure 14 defines a central longitudinal axis 20 extending through first end 16 and second end 18 and has a first annular inlet 22 positioned at first end 16 and a second annular inlet 24 positioned at second end 18. In an alternative embodiment, first annular inlet 22 is positioned at second end 18, and second annular inlet 24 is positioned at first end 16. Volumetric enclosure 14 is bounded by a perforated wall structure 30 to provide flexural strength to scaffold cage 12. Perforated wall structure 30 has an interior surface 32 and an exterior surface 34 that are connected by a set of corridors 40. Interior surface 32 defines an interior chamber 42 of sufficient volume to receive tissue-graft material, and interior surface 32 and exterior surface 34 define between them a thickness 44 of perforated wall structure 30. Corridors 40 extend through thickness 44 of perforated wall structure 30 to facilitate movement of tissue-graft material (not shown) throughout interior chamber 42, other scaffold cages, and host tissues.

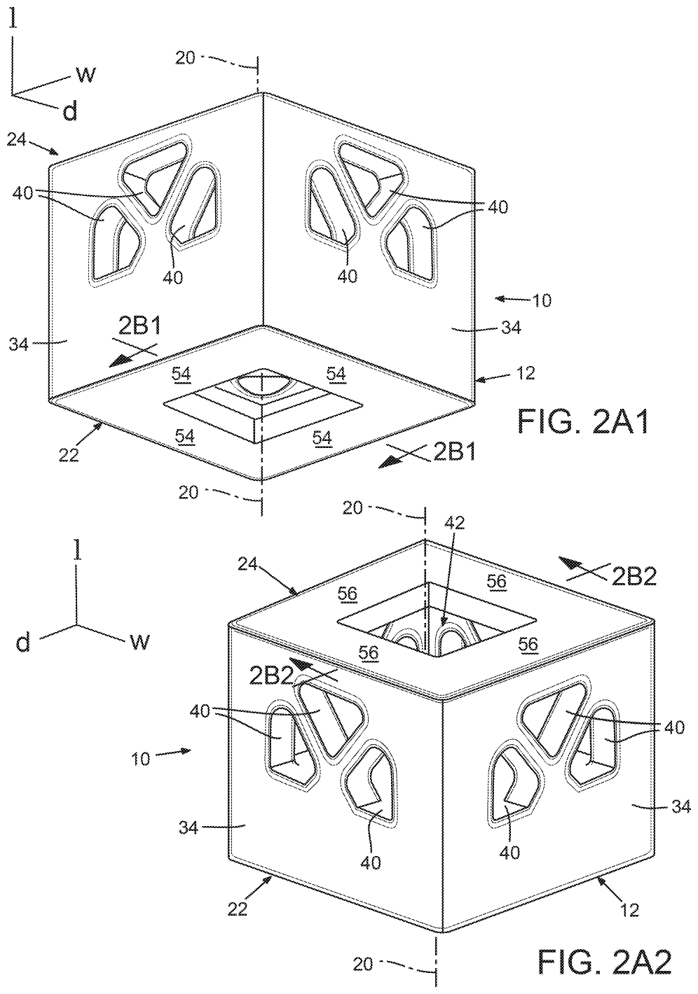

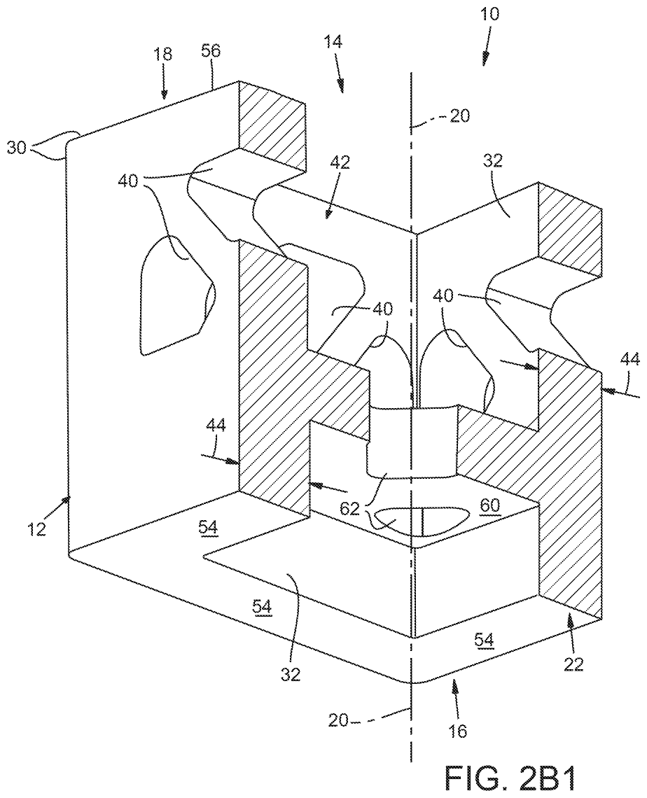

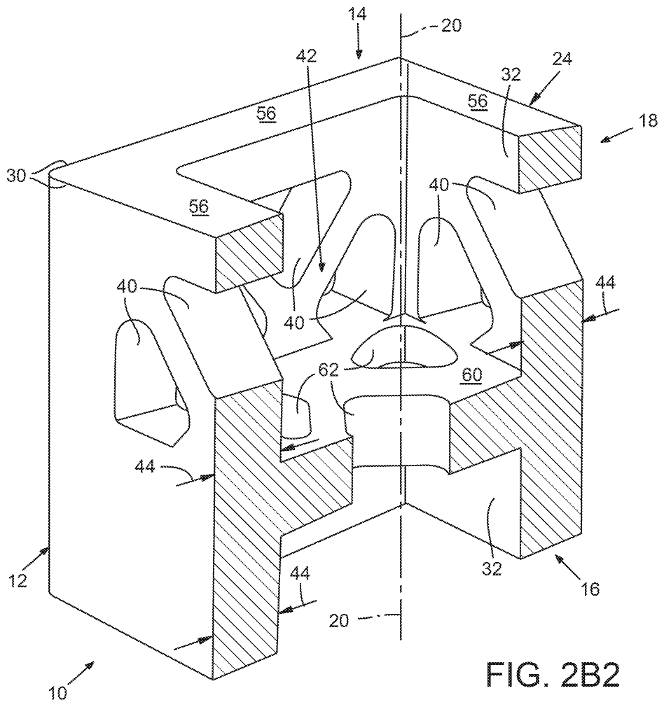

FIGS. 2A1 and 2A2 are oblique isometric views showing, respectively, a first annular inlet 22 and a second annular inlet 24. As shown in FIGS. 2A1 and 2A2, first annular inlet 22 includes a first conjoining surface 54 and second annular inlet 24 includes a second conjoining surface 56. FIGS. 2A1 and 2A2 show, first annular inlet 22 and second annular inlet 24 forming, respectively, first conjoining surface 54 and second conjoining surface 56, each of the surfaces having rectangular (specifically, square) shape and forming apertures that give access to interior chamber 42. First conjoining surface 54 and second conjoining surface 56 are each set in transverse relation to central longitudinal axis 20 and configured so that, whenever confronting annular inlets of mutually adjacent scaffold cages 12 are conjoined, confronting ones of first conjoining surface 54 or second conjoining surface 56 form complementary surfaces to each other to facilitate fusing or adhering the adjacent scaffold cages 12.

FIGS. 2B1 and 2B2 are sectional views taken, respectively, along lines 2B1-2B1 of FIG. 2A1 and 2B2-2B2 of FIG. 2A2 that show interior chamber 42, thickness 44 of perforated wall structure 30, and a perforated platform 60. In the embodiment shown, the measure of thickness 44 is generally uniform along the length of perforated wall structure 30. In some embodiments, the measure of thickness 44 varies along the length of perforated wall structure 30. In other embodiments, structural support for first annular inlet 22 and second annular inlet 24 may be provided by increasing thickness 44 at about 5 μm-1000 μm, respectively, from first annular inlet 22 and second annular inlet 24, along the length of perforated wall structure 30, relative to thickness 44 along the remaining length of perforated wall structure 30. Perforated platform 60 is set within interior chamber 42 in transverse relation to central longitudinal axis 20 and is bounded by interior surface 32. Perforated platform 60 provides support for tissue-graft material placed in interior chamber 42 and includes passageways 62 for the movement of tissue-graft material within interior chamber 42 between first end 16 and second end 18.

In some embodiments, interior chamber 42 contains tissue-graft material that supports growth of tissue. Examples of tissue-graft material include hydrogel, microgel, extracellular suspension, pharmaceutical compound, or autologous tissue. The tissue-graft material may be cell-laden or acellular. The tissue-graft material may contain cellular growth factors including Vascular Endothelial Growth Factor (VEGF), Platelet-Derived Growth Factor (PDGF), or Bone Morphogenic Protein 2 (BMP-2); be pre-vascularized; or be geometrically micropatterned.

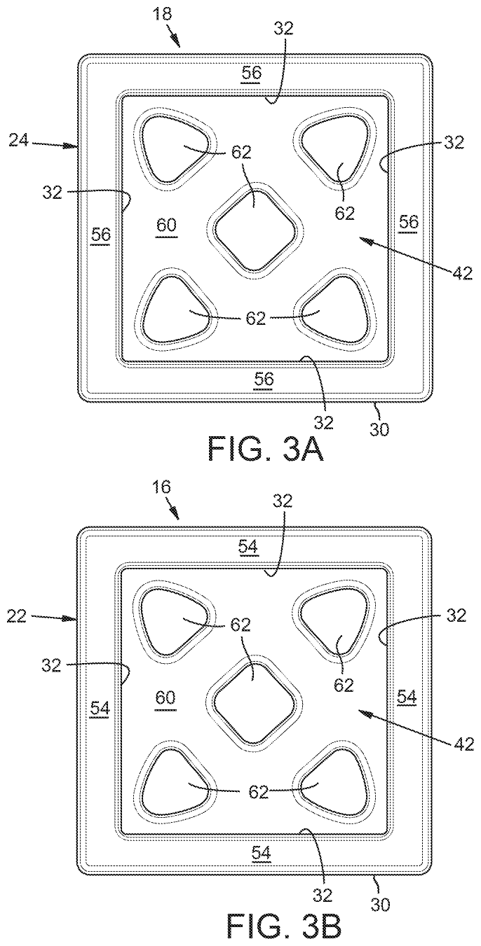

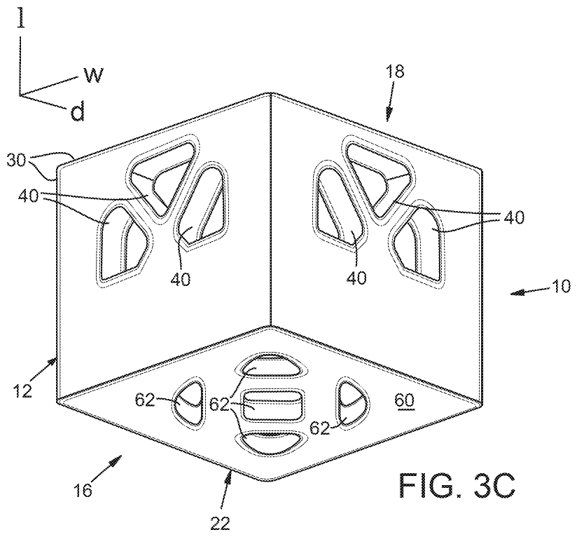

FIGS. 3A and 3B are, respectively, top and bottom plan views of scaffold cage 12 of FIGS. 2A1 and 2A2. FIG. 3A shows another view of second annular inlet 24, perforated platform 60, passageways 62, and perforated wall structure 30. FIG. 3B shows another view of first annular inlet 22 perforated platform 60, passageways 62, and perforated wall structure 30. As shown, first annular inlet 22 and second annular inlet 24 provide apertures leading to interior chamber 42 for movement of tissue-graft material within and out of scaffold cage 12. Perforated platform 60 provides passageways 52 for movement of tissue-graft material within interior chamber 42 between first end 16 and second end 18 of volumetric enclosure 18.

FIG. 3C is an oblique isometric view of alternative embodiment of a scaffold cage 12 configured have perforated platform 60 set against first annular inlet 22 of scaffold cage 12. As shown in FIG. 3C, perforated platform 60 is bounded at the margins of first end 16 and first annular inlet 22 to thereby form a flush surface of the cube. In the embodiment shown, perforated platform 60 allows for interior chamber 48 to contain tissue-graft material between first end 16 and second end 18 of volumetric enclosure 14. In other embodiments, perforated platform 60 is bounded at the margins of second end 18 and second annular inlet 24 to thereby form a flush surface of the cube.

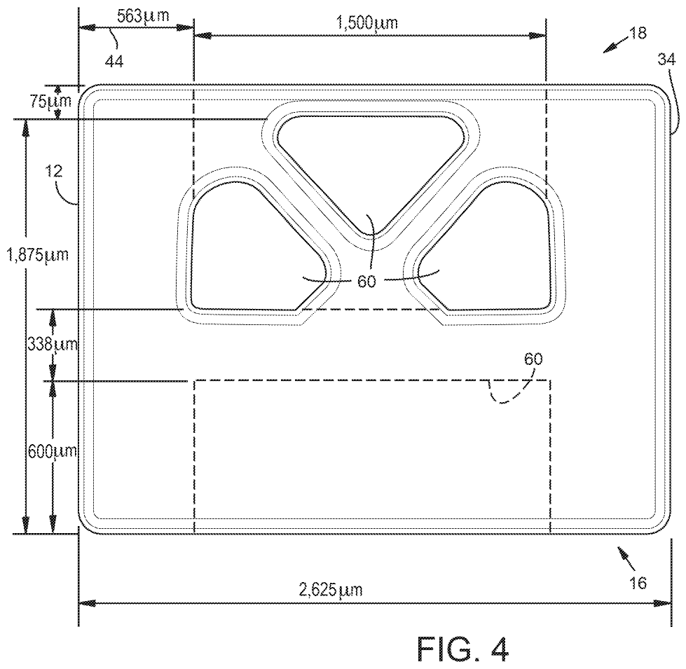

FIG. 4 is a schematic diagram showing size and feature dimensions superimposed on a side view of scaffold cage 12 of FIG. 1. The length, width, and depth dimensions of scaffold cage 12 are defined by the coordinate system shown on FIG. 4. In the embodiment shown, perforated wall structure 30 has length of 1,950 μm, width of 2,625 μm, and depth of 2,625 μm, with thickness 44 of perforated wall structure 30 set to 563 μm; perforated platform 60 has a thickness of 338 μm; first annular inlet 22 and second annular inlet 24 have respective widths of 2,065 μm and depths of 2,065 μm, and have apertures leading to interior chamber 42 of crosswise measure between about 1,500 μm-2,121 μm; corridors 40 have apertures of crosswise measure between about 1 μm-800 μm and are set in perforated wall structure 30 at 75 μm from second annular inlet 26 and adjacent to thickness 44 and perforated platform 60.

In some embodiments, the dimensions of perforated wall structure 30 range between about (1,000 μm-3,000 μm)×(1,000 μm-3,000 μm)×(1,000 μm-3,000 μm), with thickness 44 ranging between about 100 μm-645 μm. In other embodiments, perforated platform 60 has a thickness ranging between about 125 μm-400 μm and is bounded by interior surface 32. In other embodiments, first annular inlet 22 and second annular inlet 24 have dimensions ranging between about (770 μm-2315 μm)×(770 μm-2315 μm) and an aperture leading to interior chamber 42 having a crosswise measure ranging between about 500 μm-2425 μm. In some embodiments, the crosswise measure and shape of the apertures of first annular inlet 22 and second annular inlet 24 may be configured by varying the width of perforated wall structure 30 between about 5 μm-1000 μm from, respectively, first conjoining surface 54 and second conjoining surface 56. In further embodiments, corridors 40 have apertures having crosswise measures ranging between 190-915 μm and are set in perforated wall structure 30 between about 28-86 μm from either first annular inlet 22 or second annular inlet 24. These dimensional ranges are preferred to provide a therapeutically effective tissue graft scaffold of sufficient flexural strength and permeability.

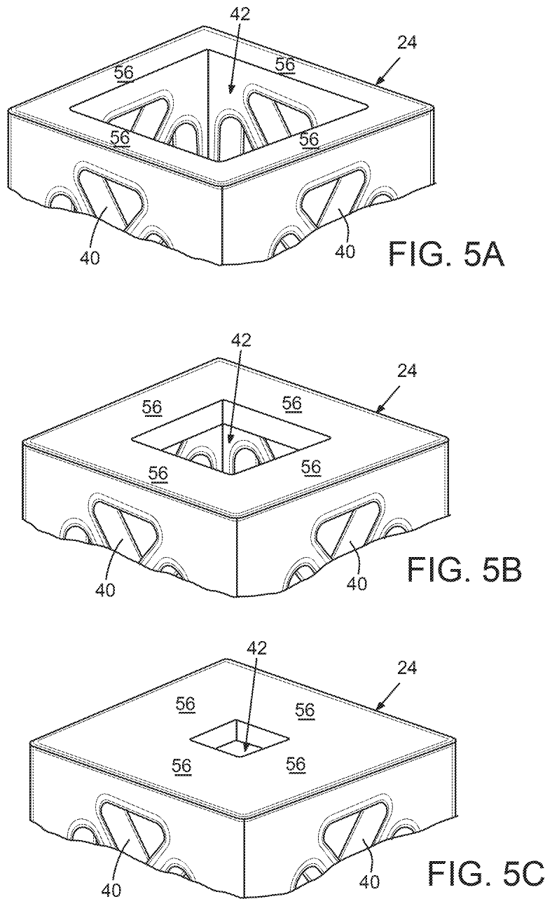

FIGS. 5A, 5B, and 5C are fragmentary oblique isometric views showing, respectively, first, second, and third alternative embodiments of second annular inlet 24. FIGS. 5A, 5B, and 5C show exemplary embodiments of second conjoining surfaces 56 of second annular inlets 24 having apertures of different cross-wise measures to provide selectable control of the movement of tissue-graft material within and out of scaffold cage 12. In other embodiments, first conjoining surface 54 of first annular inlet 22 have apertures of different cross-wise measures to provide selectable control of the movement of tissue-graft material within and out of scaffold cage 12. Structural support for first annular inlet 22 and second annular inlet 24 may be provided by increasing thickness 44 at about 5 μm-1000 μm, respectively, from first annular inlet 22 and second annular inlet 24, along the length of perforated wall structure 30, relative to thickness 44 along the remaining length of perforated wall structure 30. In a preferred embodiment, the shape and crosswise measure of first annular inlet 22 and second annular inlet 24 are generally uniform to provide structural integrity to a set of conjoined scaffold cages 12. In alternative embodiments, the shape and crosswise measure of first annular inlet 22 and second annular inlet 24 may differ. In a preferred embodiment, first conjoining surface 54 and second conjoining surface 56 have generally the same shape and surface area to provide for strength of fusion or adhesion between a set of conjoined scaffold cages 12 of tissue-graft scaffold 10. In other embodiments, first conjoining surface 54 and second conjoining surface 56 have different shapes and crosswise measures.

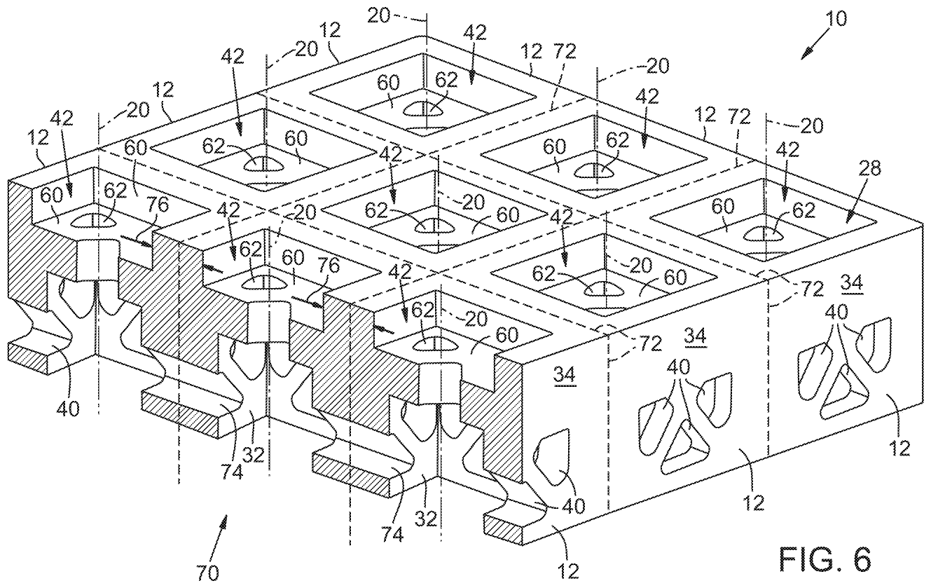

FIG. 6 is a cross-sectional isometric view of a 3×3 synthetic tissue-graft scaffold cage sheet 70 formed by fusing in a 3×3 arrangement nine replicas of the scaffold cage of FIGS. 2A1 and 2A2. “Replicas” are described herein as nominally identical in that they exhibit the same features and dimensions within manufacturing tolerances. As shown in FIG. 6, each scaffold cage 12 in scaffold cage sheet 70 has a central longitudinal axis 20, an interior surface 32, an exterior surface 34, an interior chamber 42, and a perforated platform 60. Scaffold cages 12 in scaffold cage sheet 70 are oriented such that their associated central longitudinal axes 20 are in generally parallel alignment and exterior surfaces 34 of mutually adjacent scaffold cages 12 are fused to each other to form fused perforated wall structures 72 (shown in phantom lines) and spatially aligned corridors 74. Interior surfaces 32 of mutually adjacent scaffold cages 12 define between them a fused-wall thickness 76. Spatially aligned corridors 74 extend through fused-wall thicknesses 76 of fused perforated wall structures 72 to allow migration of tissue-graft material between interior chambers 42 of mutually adjacent scaffold cages 12. Spatially aligned corridors 74 extend through fused-wall thicknesses 76 to interior chambers 42 of scaffold cage sheet 70 to facilitate movement of tissue-graft material (not shown) between interior chambers 42, other scaffold cages 12, and host tissues. Spatially aligned corridors 74 and perforated platforms 60 facilitate movement of tissue-graft material (not shown) between interior chambers 48 within scaffold cage sheet 70.

In some embodiments, a cross-sectional surface area of the apertures of individual corridors 40 or spatially aligned corridors 74 ranges between about 10,000 μm²-810,000 μm² to allow vascularization to develop within the tissue-graft material. Corridors 40 or spatially aligned corridors 74 may be of any cross-sectional shape, including circular, elliptical, or polygonal; and the apertures of corridors 40 and the crosswise dimension of spatially aligned corridors 74 may range between about 1 μm-1,000 μm.

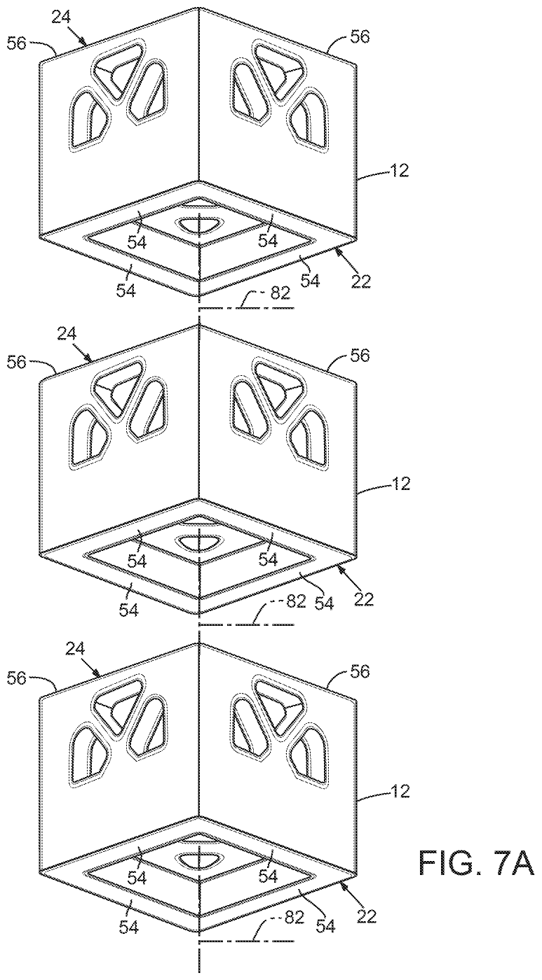

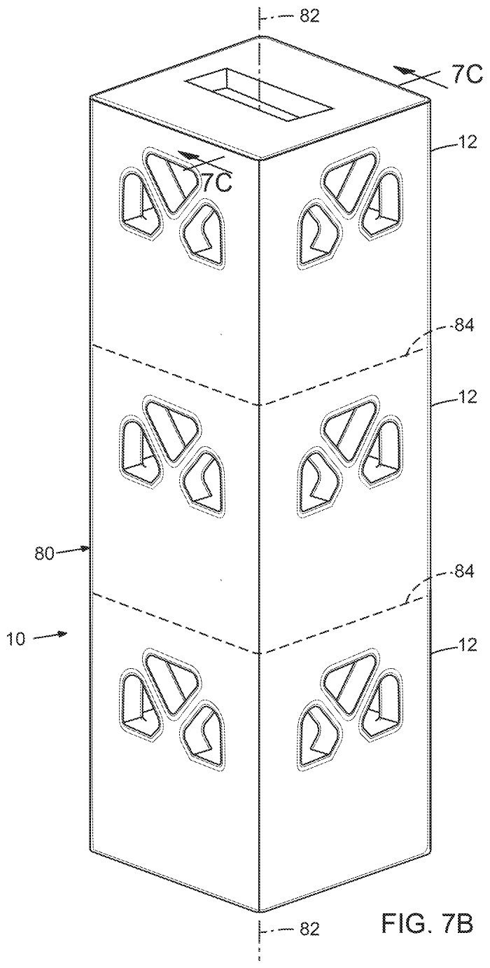

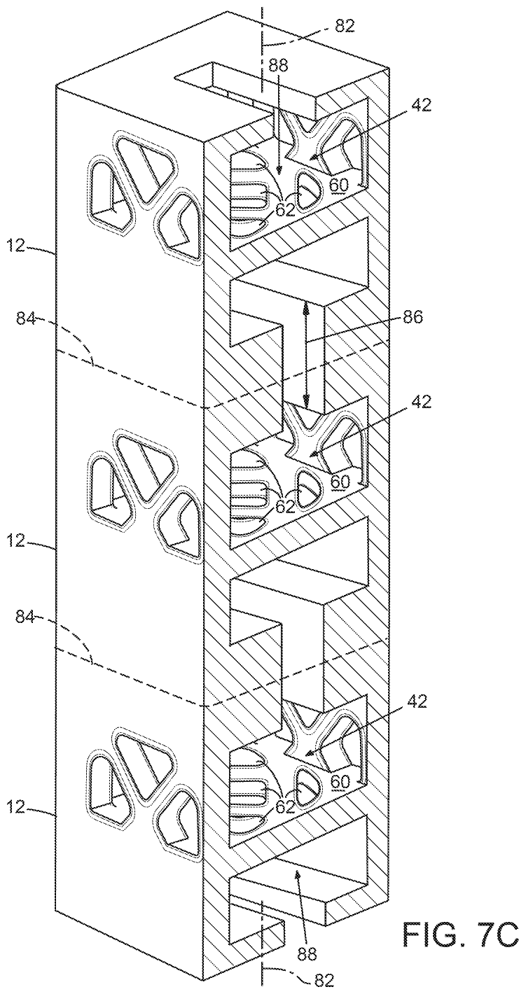

FIGS. 7A and 7B are respective exploded and isometric views of three scaffold cages 12 arranged for assembly to construct a scaffold cage tier 80. Each scaffold cage 12 in scaffold cage tier 80 includes a first annular inlet 22 at first end 16, a second annular inlet 24 at second end 18, and a volumetric enclosure 14 that defines a central longitudinal axis 20. Each first annular inlet 22 and each second annular inlet 24 of cages 12 have, respectively, a first conjoining surface 54 and a second conjoining surface 56. The associated central longitudinal axes 20 of scaffold cages 12 are arranged collinearly and define, collectively, a tier axis 82. First conjoining surface 54 and second conjoining surface 56 are each set in transverse relation to central longitudinal axis 20 and configured so that, whenever confronting annular inlets of a pair of scaffold cages 12 are conjoined to form a scaffold cage tier 80, confronting ones of first conjoining surface 54 or second conjoining surface 56 form complementary surfaces to each other to facilitate fusing or adhering the pair of scaffold cages 12. In some embodiments, first conjoining surfaces 54 may be conjoined together. In other embodiments, second conjoining surfaces 56 may be conjoined together. In a preferred embodiment, the complementary conjoining surfaces are fused by an additive manufacturing process. In some embodiments, an adhesive may be used to fasten the complementary conjoining surfaces together. For example, scaffold cage tier 80 may be assembled by selectively fusing, by an additive manufacturing process, confronting first conjoining surfaces 54 and second conjoining surfaces 56 of scaffold cages 12 aligned along tier axis 82 to form fused inlet structures 84 (shown in phantom lines).

FIG. 7C is a cross-sectional isometric view of a 3×1 scaffold cage tier 80 taken along lines 7C-7C of FIG. 7B. FIG. 7C shows each fused inlet structure 84 of scaffold cage tier 80 having a fused-inlet thickness 86 through which spatially aligned inlets extend. Fused inlet structures 84 collectively provide a tier passageway 88 to allow migration of material between the interior chambers 42 of conjoined scaffold cages 12. Fused-inlet thickness 86 may be selectively varied to provide structural integrity to cage tier 80. In a preferred embodiment, fused-inlet thickness 86 is about 150 μm. In some embodiments, fused-inlet thickness 86 ranges between about 5 μm-2000 μm.

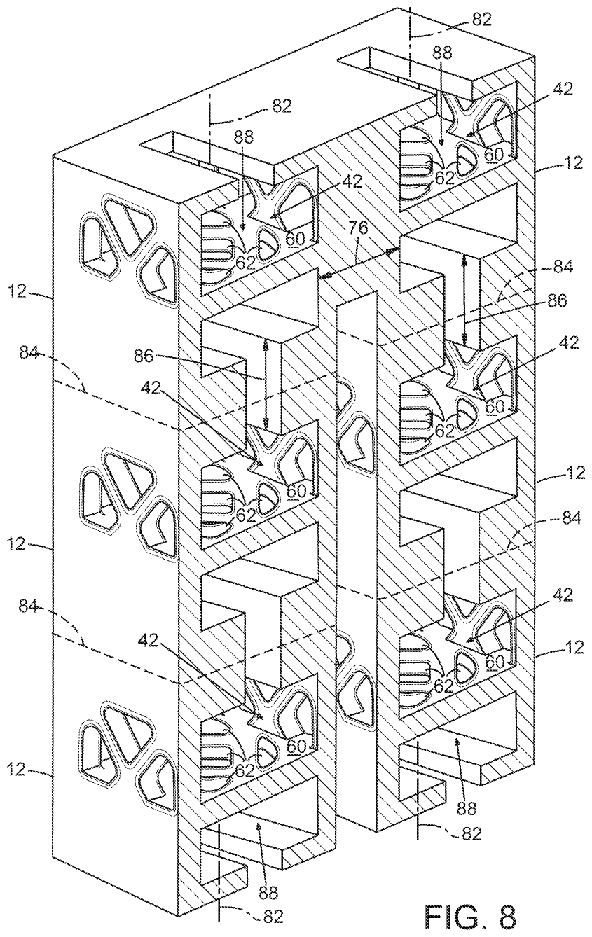

FIG. 8 is an isometric cross-sectional view of two 3×1 cage tiers 80 having a fused perforated wall structure 72 between a pair of mutually adjacent scaffold cages 12 of cage tiers 80 to form a tiered cage sheet 90. Tiered cage sheet 90 includes an array of multiple scaffold cage tiers 80 oriented such that their associated tier axes 82 are in generally parallel alignment. As shown in FIG. 8, exterior surfaces 34 of mutually adjacent scaffold cages 12 from aligned cage tiers 80 are fused together to form a fused perforated wall structure 72 between the cages. Fused perforated wall structure 72 has a fused-wall thickness 76 through which spatially aligned corridors 74 extend to allow migration of material between interior chambers 42 of the cages. In some embodiments, tiered cage sheet 90 has multiple mutually adjacent scaffold cages 12 from the array of cage tiers 80 that are fused to form multiple fused perforated wall structures 72, with each fused perforated wall structure 72 having a fused-wall thickness 76. In some embodiments, fused-wall thicknesses 76 of perforated wall structures 72 included in the array may be selectively varied to provide structural support to tiered cage sheet 90.

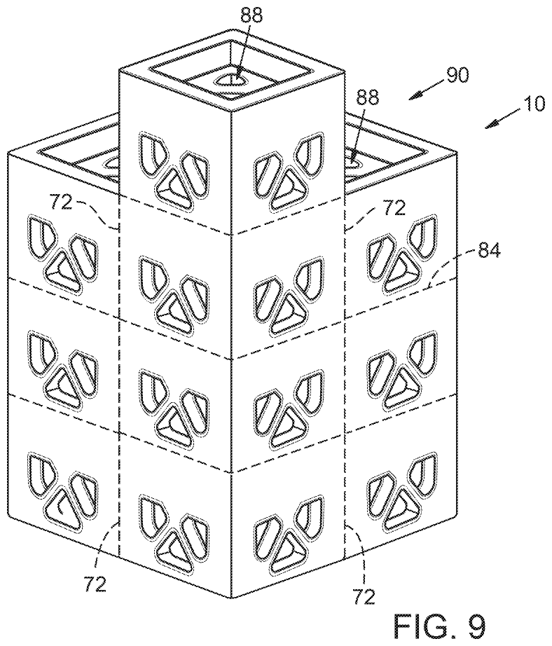

FIG. 9 shows an isometric view of one 4×1 and two 3×1 cage tiers 80 assembled to form a tiered cage sheet 90 having multiple fused perforated wall structures 72 and fused inlet structures 84 (shown in phantom lines) to form a synthetic tissue-graft scaffold 10 configured for placement into a site of repair. Tier passageways 88 extend through and communicate with the interior chambers of the volumetric enclosures 14 of the collective scaffold cages 12 of tiered cage sheet 90 to enable migration of material within and out of the scaffold cage. Synthetic tissue-graft scaffolds 10 may be selectively configured into a three-dimensional shape suitable for insertion into a site of repair.

In a preferred embodiment, the scaffold cages and scaffold cage sheets are made of β-tricalcium phosphates (β-TCP) for increasing the Ca²⁺/PO₄³⁻-dependent osteogenic signaling of human mesenchymal stem cells (hMSCs). LithaBone TCP 2000 (manufactured by Lithoz America LLC or “Lithoz”) is an example of a commercially prepared tri-calcium phosphate (Ca₃(PO₄)₂) product that is useful for bone replacement techniques. Moreover, tri-calcium phosphate materials generally are useful as bone replacement scaffolding because of their similarity to the mineral portion of human bone and have high biocompatibility, osteoconductivity, and resorbability. In some embodiments, the scaffold cages and scaffold cage sheets may be made from α-TCP, dicalcium phosphates, calcium carbonates, zirconium oxides or aluminum oxides. In other embodiments, they may be made of any material suitable for a specific function.

In a preferred embodiment, the scaffold cages and scaffold cage sheets are manufactured by lithography-based ceramic manufacturing (LCM) 3D printing technology. Examples of LCM 3D-printing instruments include the Lithoz Cera Fab 7500 and 8500 printers that have a printing resolution of about 40 μm. In one example of LCM 3D-printing, a ceramic powder (e.g., ASTM1088-04a certified β-TCP) is homogenously dispersed in a photocurable monomer and selectively polymerized via digital light projection (DLP) printing. The photolymerized slurry forms a composite of ceramic particles within a photopolymer matrix, and the organic matrix is removed via pyrolysis during sintering, which densifies the ceramic body to about 97% density. The resulting flexural strength of the printed material is about 35 MPa (similar to a trabecular bone), and its indentation modulus is generally equal to, or greater than, 100 GPa. In some embodiments, the scaffold cages and scaffold cage sheets may be manufactured using Osteoink™, which is a 3D-printable, osteoconductive calcium-phosphate material that sets in aqueous media without the need for sintering. In other embodiments, the scaffold cages and scaffold cage sheets may be manufactured by any other suitable three-dimensional printing technologies. In further embodiments, they may be made by any mold-based (such as reaction injection molding), sculpting-based, or subtractive manufacturing methods.

It will be obvious to those having skill in the art that many changes may be made to the details of the above-described embodiments without departing from the underlying principles of the invention. For example, first annular inlet 22 and second annular inlet 24 of a scaffold cage 12 can be of other than square shape. Other possible shapes include an ellipse, a triangle, other polygons, or a circle. The scope of the present invention should, therefore, be determined only by the following claims.

1. In a synthetic tissue-graft scaffold including a set of one or more nominally identical scaffold cages that are configured to facilitate regrowth of tissue of an organism in and around the scaffold cages, each one of the scaffold cages in the set comprising:
a volumetric enclosure bounded by a perforated wall structure and having interior and exterior surfaces and first and second opposite ends, the volumetric enclosure defining a central longitudinal axis that extends through the first and second opposite ends, the interior surface defining a boundary of an interior chamber of the volumetric enclosure, and the interior and exterior surfaces defining between them a thickness of the perforated wall structure;
first and second annular inlets positioned at, respectively, the first and second ends of the volumetric enclosure, the first and second inlets forming, respectively, first and second conjoining surfaces that are transverse to the central longitudinal axis and configured so that, whenever confronting annular inlets of a pair of the scaffold cages in the set are conjoined, confronting ones of the first or second conjoining surfaces of the pair of scaffold cages form complementary surfaces to each other;
a perforated platform bounded by the interior surfaces of the volumetric enclosure and set in transverse relation to the central longitudinal axis, the perforated platform providing a passageway within the interior chamber of the volumetric enclosure between its first and second opposite ends; and
corridors extending through the thickness of the perforated wall structure and communicating with the passageway within the interior chamber of the volumetric enclosure to enable migration of material within and out of the scaffold cage.

2. The synthetic scaffold of claim 2, in which the first and second annular inlets are positioned at, respectively, the second and first opposite ends.

3. The synthetic scaffold of claim 1, in which the set includes an array of multiple nominally identical scaffold cages in the form of a scaffold cage sheet, the multiple scaffold cages oriented such that their associated central longitudinal axes are in generally parallel alignment and the exterior surfaces (20) of mutually adjacent cages are fused to each other and thereby form a fused perforated wall structure, the fused perforated wall structure having a fused-wall thickness through which spatially aligned corridors extend to allow migration of material between the interior chambers of the mutually adjacent scaffold cages.

4. The synthetic scaffold of claim 1, in which the set includes an array of multiple nominally identical scaffold cages in the form of a scaffold cage tier, the multiple scaffold cages oriented such that their associated central longitudinal axes are collinear and define, collectively, a tier axis, and confronting ones of first and second conjoining surfaces of scaffold cages aligned along the tier axis are fused to each other and thereby form a fused inlet structure, the fused inlet structure having a fused-inlet thickness through which spatially aligned annular inlets extend, the aligned annular inlets collectively providing a tier passageway to allow migration of material between the interior chambers of conjoined scaffold cages.

5. The synthetic scaffold of claim 4, in which the set includes an array of multiple scaffold cage tiers in the form of a tiered cage sheet, the multiple cage tiers oriented such that their associated tier axes are in generally parallel alignment and the exterior surfaces of a pair of mutually adjacent scaffold cages from the aligned cage tiers are fused to each other and thereby form a fused perforated wall structure between the pair, the fused perforated wall structure having a fused-wall thickness through which spatially aligned corridors extend to allow migration of material between the interior chambers of the pair of scaffold cages.

6. The synthetic scaffold of claim 5, in which the exterior surfaces of multiple mutually adjacent scaffold cages from the aligned cage tiers are fused to each other and thereby form fused perforated wall structures, each fused perforated wall structure having a fused-wall thickness through which spatially aligned corridors extend to allow migration of material between the interior chambers of the fused scaffold cages.

7. The synthetic scaffold of claim 1, in which the synthetic scaffold is made of β-tricalcium phosphate.

8. The synthetic scaffold of claim 1, in which the synthetic scaffold is made of α-tricalcium phosphate, dicalcium phosphate, calcium carbonate, zirconium oxide, or aluminum oxide.

9. The synthetic scaffold of claim 1, in which the synthetic scaffold is manufactured using a lithography-based three-dimensional printing technology.

10. The synthetic scaffold of claim 1, in which the synthetic scaffold is manufactured using a mold-based, a sculpting-based, or a subtractive manufacturing method.

11. The synthetic scaffold of claim 1, in which the first conjoining surface of the first annular inlet is generally shaped as a circle, ellipse, or polygon.

12. The synthetic scaffold of claim 1, in which the second conjoining surface of the second annular inlet is generally shaped as a circle, ellipse, or polygon.

13. The synthetic scaffold of claim 1, in which the perforated platform constitutes a first perforated platform, and further comprising a second perforated platform, the second perforated platform set transverse to the central longitudinal axis of the volumetric enclosure of the cage and proximal to the second end of the volumetric enclosure relative to the first perforated platform to define a platform pair, the platform pair providing a passageway within the interior chamber of the volumetric enclosure between the first and second ends.

14. The synthetic scaffold of claim 1, in which the exterior surface of the perforated wall structure constitutes one or more wall aspects, and the perforated wall structure includes no corridor extending through its fused-wall thickness at one or more of the wall aspects.

15. The synthetic scaffold of claim 1, in which the passageway within the interior chamber terminates at and therefore does not extend through one of the first and second opposite ends of the volumetric enclosure.

16. The synthetic scaffold of claim 1, further comprising a tissue-graft material inserted into the interior chamber of the volumetric enclosure.
